(19) United States
(12) Patent Application Publication
(10) Pub. No.: US 2025/0277737 A1
(43) Pub. Date: Sep. 4, 2025

(54) INTERLEAVE-SAMPLED PHOTOACOUSTIC (PA) IMAGING

(71) Applicant: THE REGENTS OF THE UNIVERSITY OF CALIFORNIA, Oakland,, CA (US)

(72) Inventors: Lei FU, La Jolla, CA (US); Jesse Vincent JOKERST, La Jolla, CA (US)

(21) Appl. No.: 18/857,357

(22) PCT Filed: Apr. 26, 2023
(86) PCT No.: PCT/US2023/019949

§ 371 Date: Oct. 16, 2024

Related U.S. Application Data
(60) Provisional application No. 63/334,788, filed on Apr. 26, 2022.

(51) Int. Cl. G01N 21/17

(52) U.S. Cl. CPC G01N 21/1702; G01N 2021/1757

(57) ABSTRACT

In accordance with a method of obtaining at least one photoacoustic (PA) image of a sample, a plurality of light pulses is generated that cause ultrasonic emission from the sample. Ultrasonic emission signals from the sample caused by each of the light pulses are received. The ultrasonic emission signals are sampled to obtain sampled data values. A time duration between a time at which the light pulses are generated and a time at which the sampling of the ultrasonic emission signals is performed is modulated. At least one photoacoustic image of the sample is reconstructed from the sampled data values of the ultrasonic emission signals.

### CROSS REFERENCE TO RELATED APPLICATION

This application claims the benefit of U.S. Provisional Application Ser. No. 63/334,788, filed Apr. 26, 2022, the contents of which are incorporated herein by reference.

### GOVERNMENT FUNDING

This invention was made with government support under AG065776, DE029025, OD021821, awarded by the National Institutes of Health (NIH). The government has certain rights in the invention.

### BACKGROUND

High-frequency photoacoustic (PA) tomography (e.g., greater than 20 MHz) is playing an important role in peripheral biomedical applications where high-resolution is more important than penetration depth such as periodontal imaging, small vessels, and skin. In order to capture the high-frequency PA signal, an ultrasound data acquisition (DAQ) should have commensurately high sampling rate, but this leads to extensive hardware and cost limitations. Nyquist sampling theory suggests that the acquirable frequency component of a signal is less than half the DAQ's sampling rate, i.e., the Nyquist limit. For example, a DAQ with 60-MHz sampling rate can acquire signal less than 30 MHz, which can cover 100% bandwidth of a 20-MHz transducer (10 to 30 MHz). Unfortunately, an ultrasound DAQ with a high-sampling rate (>60 MHz) and massively parallel channels can be extremely expensive. The Nyquist limit applies to these DAQ systems as well. Many PA groups use clinical ultrasound DAQs, which are low cost but also have a low sampling rate, e.g., the iu22 system's sampling rate is 32 MHz. These lower sampling rates can negatively impact the development of high-frequency PA imaging.

### SUMMARY

In one aspect, an interleave-sampled PA imaging technique is described herein that enables high-frequency imaging with a relatively low sampling rate, e.g., a 41.67-MHz sampling rate with a 30-MHz transducer. In one particular embodiment, the technique harnesses two acquisitions at a low sampling rate to effectively double the sampling rate which consequently reduces the frame rate by a factor of two. In this technique the temporal offset of alternating light pulses with respect to the sampling time is modulated and can thus be applied to any PA DAQ system. Both phantom and in vivo studies were performed with a 30-MHz transducer. The results demonstrate that interleaved sampling in 41.67 MHz can capture high frequency information above 30 MHz but 41.67-MHz sampling rate cannot. The axial and lateral resolution are as high as 63 μm and 91 μm via interleaved sampling which are much higher than those of conventional 41.67-MHz sampling (130 μm and 136 μm).

In another aspect, a method is presented of obtaining at least one photoacoustic (PA) image of a sample. In accordance with the method, a plurality of light pulses is generated that cause ultrasonic emission from the sample. Ultrasonic emission signals from the sample caused by each of the light pulses are received. The ultrasonic emission signals are sampled to obtain sampled data values. A time duration between a time at which the light pulses are generated and a time at which the sampling of the ultrasonic emission signals is performed is modulated. At least one photoacoustic image of the sample is reconstructed from the sampled data values of the ultrasonic emission signals.

In accordance with one embodiment, the ultrasonic emission signals are sampled at a sampling rate slower than a Nyquist limit.

In accordance with another embodiment, the modulating includes offsetting alternating light pulses from an immediately preceding pulse by a time period less than a sampling period.

In accordance with another embodiment, the offset is one half of the sampling period.

In accordance with another embodiment, the offset is one third of the sampling period.

In accordance with another embodiment, the offset is one fourth of the sampling period.

In accordance with another embodiment, the plurality of light pulses is generated by an optical source, the modulating being performed by adjusting a time at which a triggering signal is sent to the optical source.

In accordance with another embodiment, the plurality of light pulses is generated by a laser source.

In accordance with another embodiment, the modulating includes adjusting a timing at which the ultrasonic emission signals are sampled.

In accordance with another embodiment, reconstructing the at least one photoacoustic image includes increasing an axial and lateral resolution of the at least one photoacoustic image relative to a photoacoustic image obtained without modulating the time duration such that the light pulses that are generated are equally spaced in time from one another.

In accordance with another embodiment, the modulating includes offsetting in time, by a time period less than a sampling period, a subset of the light pulses relative to the time at which the sampling of the ultrasonic signals is performed.

In accordance with another embodiment, the offsetting includes advancing in time the subset of the light pulses relative to the time at which the sampling of the ultrasonic signals is performed.

In accordance with another embodiment, the offsetting includes delaying in time the subset of the light pulses relative to the time at which the sampling of the ultrasonic signals is performed.

In accordance with another embodiment, the offsetting includes advancing in time a first subset of the light pulses relative to the time at which the sampling of the ultrasonic signals is performed and delaying in time a second subset of the light pulses relative to the time at which the sampling of the ultrasonic signals is performed.

In accordance with another embodiment, different ones of the light pulses in the subset of the light pulses are offset by different fractions of the sampling period.

In accordance with another embodiment, the subset of the light pulses are alternating ones of the light pulses.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. Furthermore, the claimed subject matter is not limited to implementations that solve any or all disadvantages noted in any part of this disclosure.

### BRIEF DESCRIPTION OF THE DRAWINGS

FIGS. 1a-1c illustrate the process of interleaved sampling to double the equivalent sampling rate for photoacoustic (PA) imaging.

FIGS. 2a-2i show the results of performing PA images on nichrome wires at different sampling rates.

FIGS. 3a-3c show data concerning the axial and lateral resolutions of the nichrome wires at three different sampling rates.

FIG. 4a shows in vivo imaging of a human finger at different sampling rates and FIG. 4b shows an ultrasound image of the human finger.

FIG. 5 shows the experimental setup of a phantom study in which six nichrome wires were put into a 3D-printed holder.

FIGS. 6a-6f present data comparing the in vivo results under 41.67-MHz sampling and interleaved 41.67-MHz sampling.

FIG. 7 shows a simplified schematic diagram of one example of a PA imaging system in which the methods described herein may be employed.

FIGS. 8a and 8b each show a timing diagram illustrating the laser pulses, the resulting photoacoustic signals, the time at which the photoacoustic signals are sampled, and the sampled data points that are obtained when conventional sampling is employed (FIG. 8a) and when the interleaved sampling technique described herein is employed (FIG. 8b) by advancing alternating light pulses in time relative to the time at which the resulting PA signal is sampled.

FIGS. 9a and 9b each show a timing diagram illustrating the laser pulses, the resulting photoacoustic signals, the time at which the photoacoustic signals are sampled, and the sampled data points that are obtained when conventional sampling is employed (FIG. 9a) and when the interleaved sampling technique described herein is employed (FIG. 9b) by delaying alternating light pulses in time relative to the time at which the resulting PA signal is sampled.

### DETAILED DESCRIPTION

Described herein are methods and systems for performing photoacoustic (PA) imaging with a relatively low sampling rate. The methods and systems employ a technique referred to herein as interleave-sampled PA imaging, which can be used to perform high-frequency imaging with a relatively low sampling rate. As described in C. Vogel, and H. Johansson, “Time-interleaved analog-to-digital converters: Status and future directions,” Ieee Int Symp Circ S, 3386-3389 (2006), interleaved sampling has improved the sampling rate in analog-to-digital conversion in signal processing. This concept is applied herein to PA imaging to e.g., double, the equivalent sampling rate. Unlike interleaved sampling in ultrasound imaging, the methods and systems described herein perform interleaved sampling by modulating the delay of the light pulses. Thus, the techniques described herein can be used with any PA data acquisition (DAQ) system because the interleaved sampling is achieved by adjusting the laser delay. No complex DAQ circuits are needed.

Although the illustrative examples of the methods and systems described herein are directed to doubling the equivalent sampling rate for PA imaging, higher multiplied sampling rates (>2×) may also be determined using this technique. As described in more detail below, this approach was validated by performing high-resolution PA imaging using a 30-MHz transducer but with only a 41.67-MHz sampling rate. To clarify, interleave-sampled PA imaging is quite different from the interleaved dual PA/ultrasound: The former improves the sampling rate whereas the latter offers dual-mode imaging.

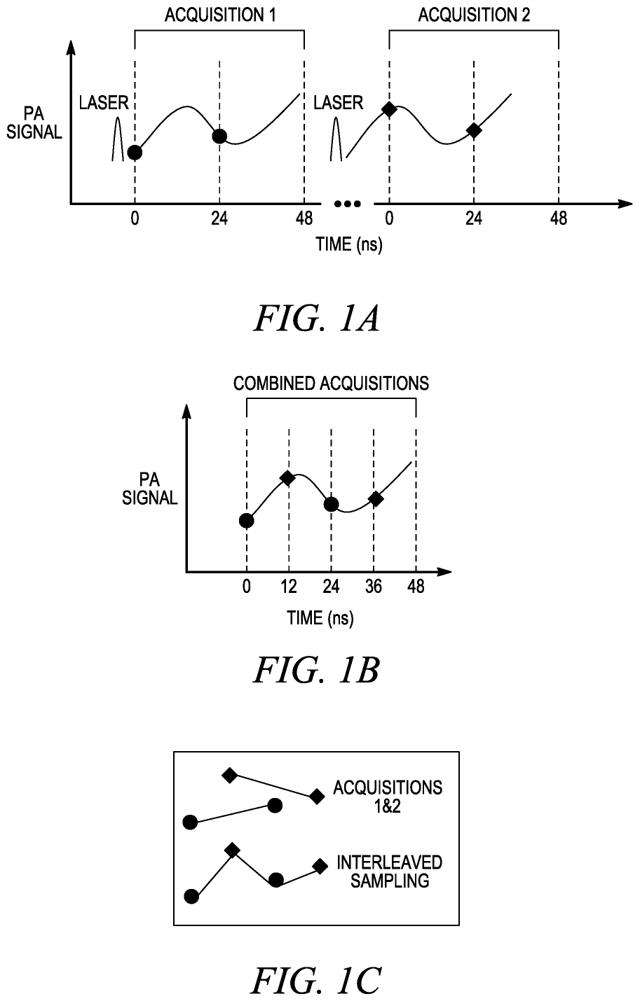

Interleaved sampling combines multiple regular acquisitions to reach an equivalently multiplied sampling rate. For example, FIG. 1(a) shows two repeated laser pulses inducing two identical PA signals subsequently. They were acquired at 41.67 MHz with a sampling period of 24 ns (1 second/41.67 MHZ=24 ns). There is only one but important difference between the two acquisitions: They start sampling at different phases of the PA signal. Specifically, in the second acquisition, the laser pulse and hence the PA signal starts 12 ns (half of the sampling period, 24 ns/2=12 ns) earlier than its sampling. Thus, the two acquisitions record the identical PA signal from different phases including two sampling dots in Acquisition 1, and two dots in Acquisition 2 (FIG. 1(a)) Interestingly, a combination of these two acquisitions (FIG. 1(b)) can provide four samplings at different phases of the PA signal as if the signal is sampled at 83.33 MHz with a sampling period of 12 ns (83.33 MHz=41.67 MHz*2; 12 ns=1 second/83.33 MHz). FIG. 1(c) compares the PA signal collected with 41.67-MHz single sampling (Acquisitions 1 & 2) and interleaved sampling at 41.67 MHz. Obviously, neither Acquisition 1 nor Acquisition 2 can fully reveal the fluctuations of the PA signal. In comparison, interleaved sampling provides four sampling events and more accurately depicts the curve of the PA signal. In other words, interleaved sampling captures more high-frequency PA signal.

It is important to ensure that the two combined acquisitions are starting at different phases of the PA signal. In some embodiments the difference should be half of the sampling period. However, in other embodiments the difference can be by any suitable amount, including, without limitation, one third or one fourth of the sampling period. One can delay the data acquisition with respect to the light pulse, which might require complex DAQ circuits. In the particular implementation illustrated herein, the PA signal is delayed by delaying the laser's Q-switch trigger and hence the light pulse and the PA signal for interleaved sampling. Alternatively, modulating the traveling distance of laser pulse in a fiber can be used to delay the laser pulse by nanoseconds.

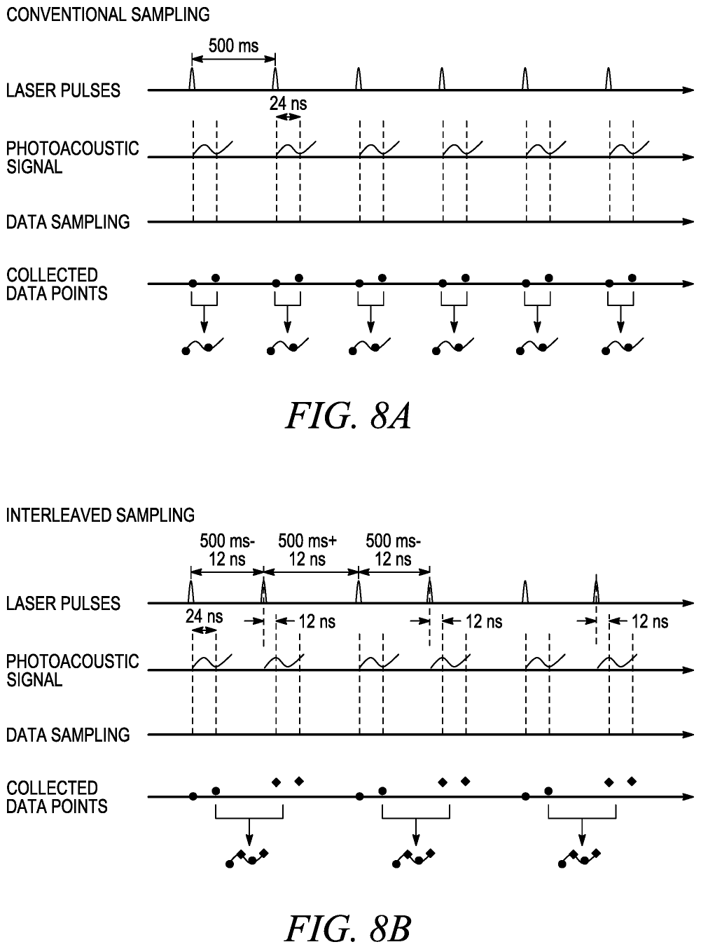

In the example of the interleaved sampling technique described above and illustrated in FIG. 1 the light pulse in the second acquisition is advanced relative to the time at which the resulting PA signal is sampled. This is further illustrated in FIGS. 8a and 8b, where FIG. 8a shows the conventional sampling technique in which in which the light pulses are equally spaced in time from one another and FIG. 8b shows the interleaved sampling technique described herein in which alternating light pulses are advanced in time relative to the time at which the resulting PA signal is sampled.

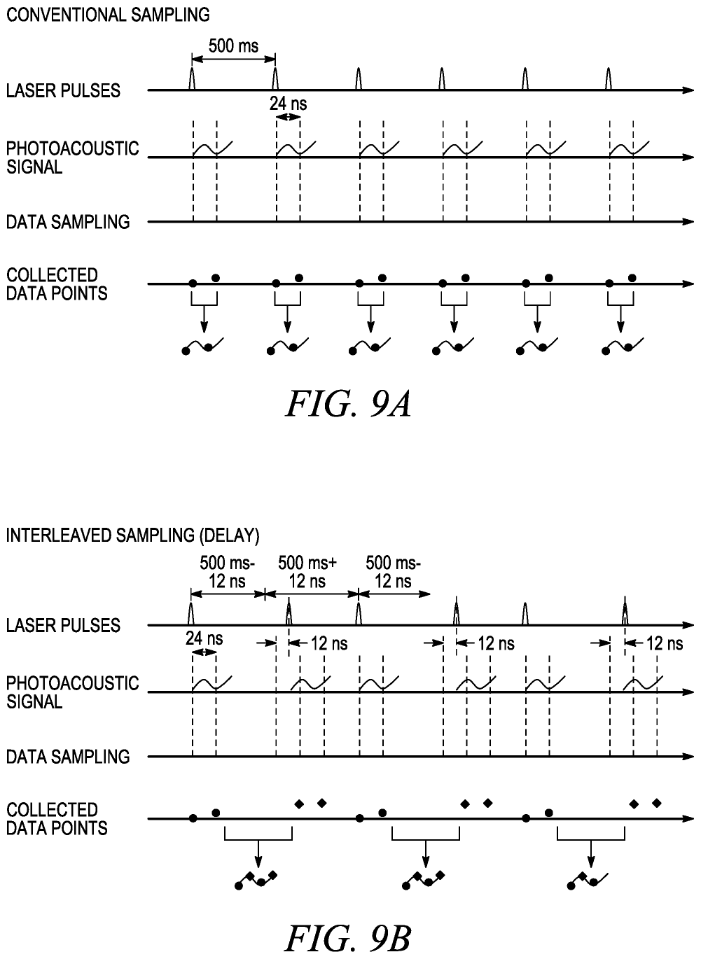

In an alternative embodiment, instead of advancing alternating light pulses in time relative to the time at which the resulting PA signal is sampled, alternating light pulses may be delayed in time relative to the time at which the resulting PA signal is sampled. This embodiment is illustrated in connection with FIGS. 9a and 9b, where FIG. 9a, like FIG. 8a, shows the conventional sampling technique in which the light pulses are equally spaced in time from one another and FIG. 9b shows the interleaved sampling technique described herein in which alternating light pulses are delayed in time relative to the time at which the resulting PA signal is sampled. Thus, FIGS. 8 and 9, taken together, illustrate that the interleaved sampling techniques described herein encompass offsetting the light pulses in time relative to the time at which the resulting PA signal is sampled, either by advancing alternating light pulses in time or by delaying alternating light pulses in time.

In yet another alternative embodiment, interleaved sampling may be performed which employs both light pulses that are advanced in time and delayed in time relative to the time at which the resulting PA signal is sampled. That is, a series of light pulses may be used in which some subset of those light pulses are advanced in time and others that are delayed in time. In some cases the different light pulses may be offset (i.e., advanced or delayed) in time relative to the time at which the resulting PA signal is sampled by different fractions of the sampling period. In this way a greater number of different samples of the PA signal can be obtained, thereby allowing a more accurate reconstruction of the PA signal.

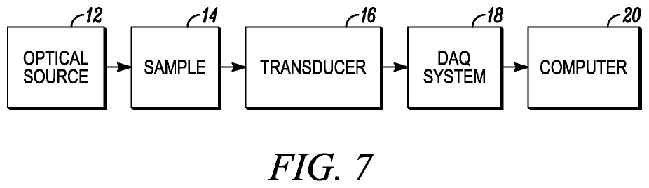

To illustrate the methods described herein, both ex vivo phantom and in vivo human experiments were performed to evaluate the interleaved sampling technique in PA imaging. For these purposes a Vantage data acquisition system was used with 256 parallel channels to receive, process, and reconstruct the PA signal (Verasonics, Inc., Kirkland, WA, USA). The maximum sampling rate is 62.5 MHz. The Vantage output trigger works as a Q-switched trigger and fires the Q-switch laser while acquiring data. This time of laser firing can be delayed in steps of 4 ns by programming the trigger [14]. A commercially available transducer (LZ400; Visualsonics, Inc., Canada) received the PA signal. Its central frequency is 30 MHz with bandwidth of 18-38 MHz and 256 elements. A tunable OPO laser (OPOTek) was the light source and operated at 20 Hz. The wavelength was fixed at 690 nm for phantom studies and 850 nm for in vivo imaging. The pulse width is 5-7 ns, and the pulse energy is 26 mJ, which is well under the laser safety limit. Nichrome heater wires (30-μm diameter) were the sample in the phantom study. The finger of a healthy adult was imaged for the in vivo study. All work with human subjects was approved by the UCSD IRB and conducted according to the ethical standards set forth by the IRB and the Helsinki Declaration of 1975. The participant gave written informed consent. Of course, more generally, any suitable PA imaging system and DAQ system may be employed. A simplified example of such a system is shown in FIG. 7.

In the illustrative embodiment PA imaging was performed at the sampling rates of 1) single sampling at 41.67 MHZ, 2) interleaved sampling in 41.67 MHZ (83.33 MHz equivalently), and 3) single sampling at 62.5 MHz. We chose these sampling rates because 41.67-MHz can barely cover the bandwidth of our 30-MHz transducer (18 MHz-38 MHz), and the interleaved sampling in 41.67 MHZ (83.33 MHz) is expected to cover the transducer bandwidth. 62.5-MHz is the maximum sampling rate of our DAQ.

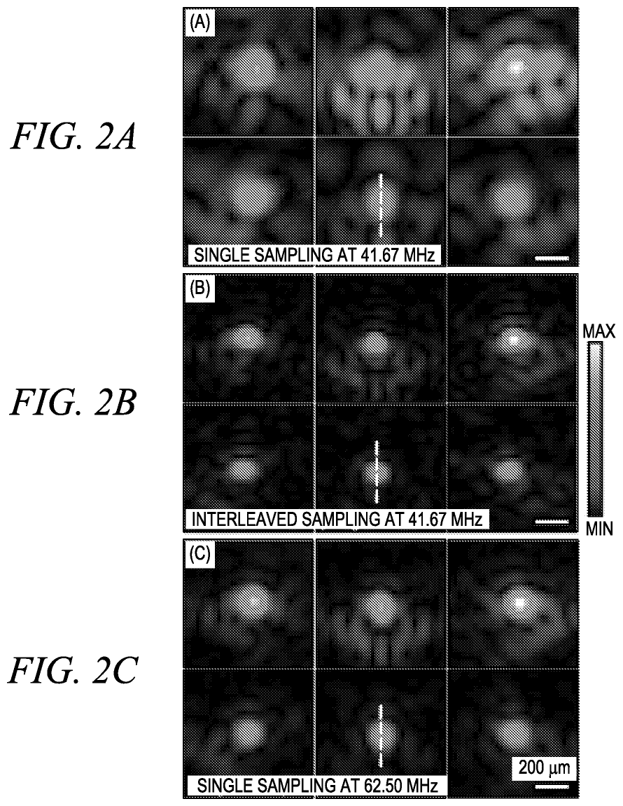

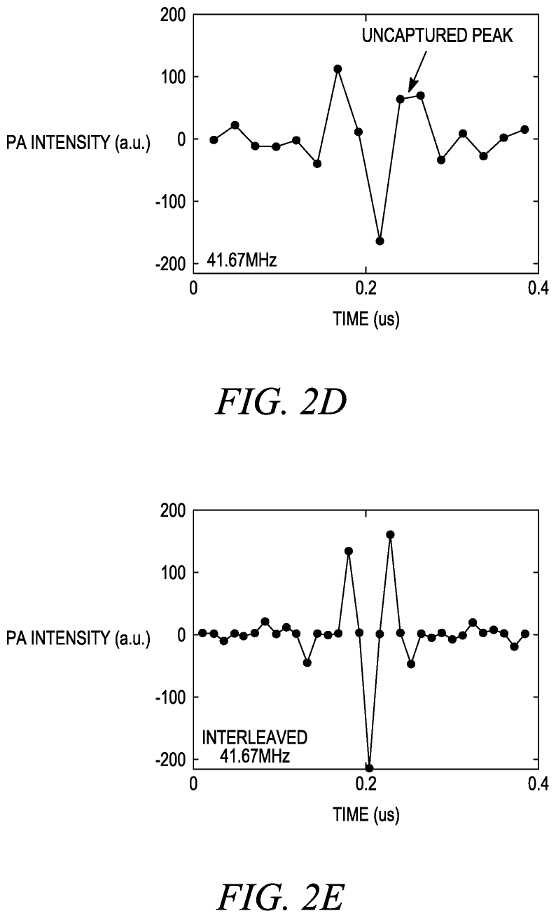

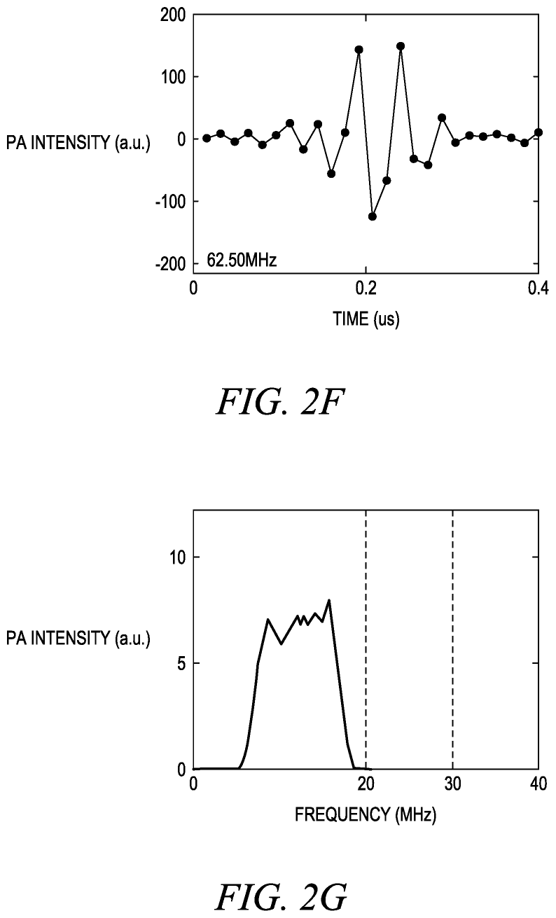

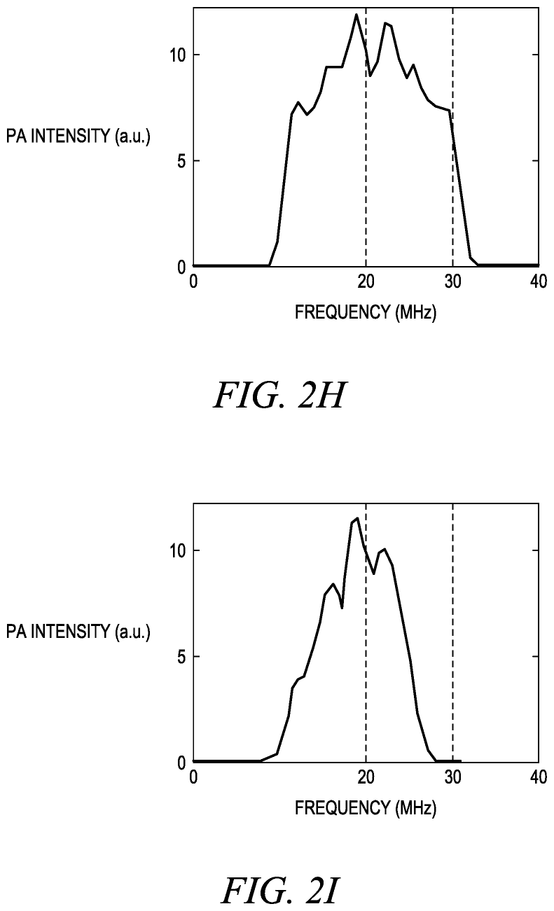

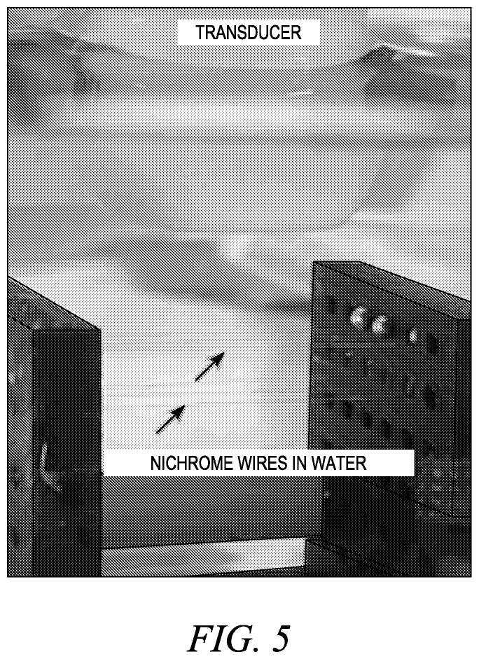

The imaging sample employed to illustrate the methods described herein were six nichrome heater wires (30 μm diameter) as the imaging sample. These wires were put into a 3D-printed holder that keeps them apart in parallel in a range of 10 mm (width)×5 mm (depth) in water, and 12 mm underneath the transducer (FIG. 5). We defined the FWHM (full width at half maximum) of the lateral and axial amplitude distributions of the wires as the lateral and axial resolution, respectively. FIGS. 2(a), 2(b) and 2(c) shows the PA images of the wire phantom acquired with single sampling at 41.67 MHZ, interleaved sampling at 41.67 MHZ, and single sampling at 62.5 MHz. All of these images are in a cross-sectional view.

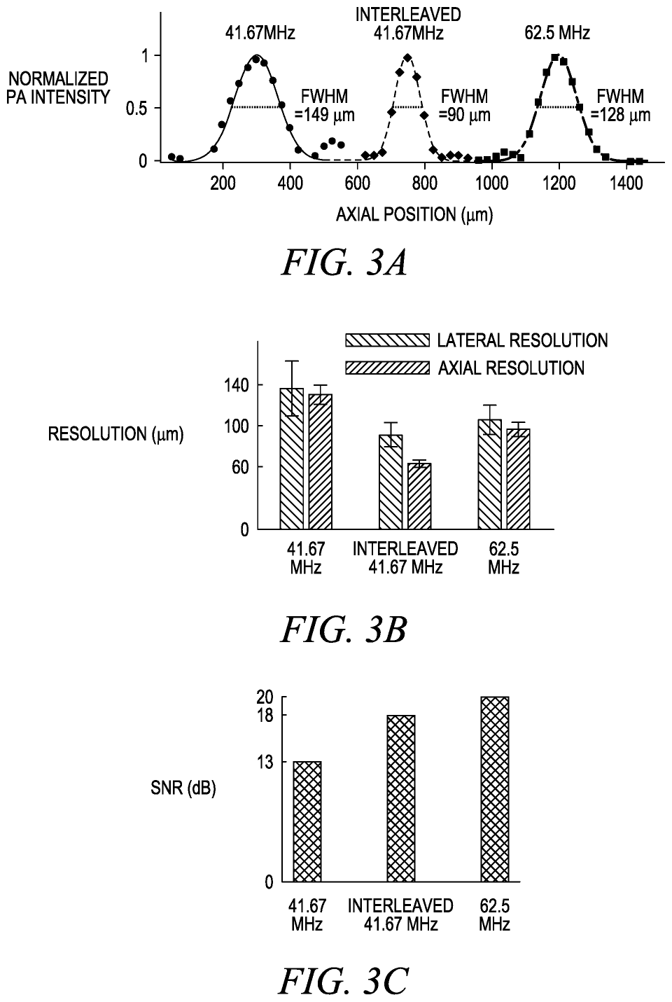

Obviously, the spots are much smaller in FIG. 2(b) than in FIG. 2(a) indicating the resolution of PA imaging is much higher by the interleaved 41.67-MHz sampling. FIG. 3(a) compares the axial amplitude distributions of the middle and bottom wire (FIG. 2) by the three different sampling rates. Note that the axial resolution is equal to FWHM minus 30 μm because the wire diameter is 30 μm. We measured the lateral and axial resolution of FIGS. 2(a), 2(b) and 2(c) by measuring the FWHM of the six wires as results shown in FIG. 3(b). The averaged axial and transverse resolution are 63 μm and 91 μm under interleaved sampling—the axial resolution is two-fold higher than that of 41.67-MHz sampling (130 μm). Most importantly, interleaved sampling at 41.67 MHz provides even better resolution than 62.5-MHz single sampling (FIGS. 2(c) and 3(b)) (maximum sampling rate of our DAQ). The Signal-to-Noise Ratio (SNR) by interleaved 41.67-MHz sampling is also higher than that of 41.67-MHz sampling (FIG. 3(c)).

FIGS. 2(d), 2(e), and 2(f) show the 1D PA signals of a single nichrome wire under the three sampling rates Interleaved sampling provides more data points, which in turn lead to more details about the PA signal than 41.67-MHz sampling. The 41.67-MHz sampling could not identify the third peak of the PA signal. We further performed fast Fourier transform (FFT) to convert 1D PA signals to the spectral domain (FIGS. 2(g), 2(h), and 2(i)). Obviously, interleaved sampling at 41.67 MHz provides a much broader high-frequency spectrum (>20 MHz) than the 41.67-MHz sampling, which has spectrum below 20 MHz. Our phantom results prove that interleave-sampled PA imaging can acquire high-frequency PA signal at a relatively low sampling rate, which improves the resolution.

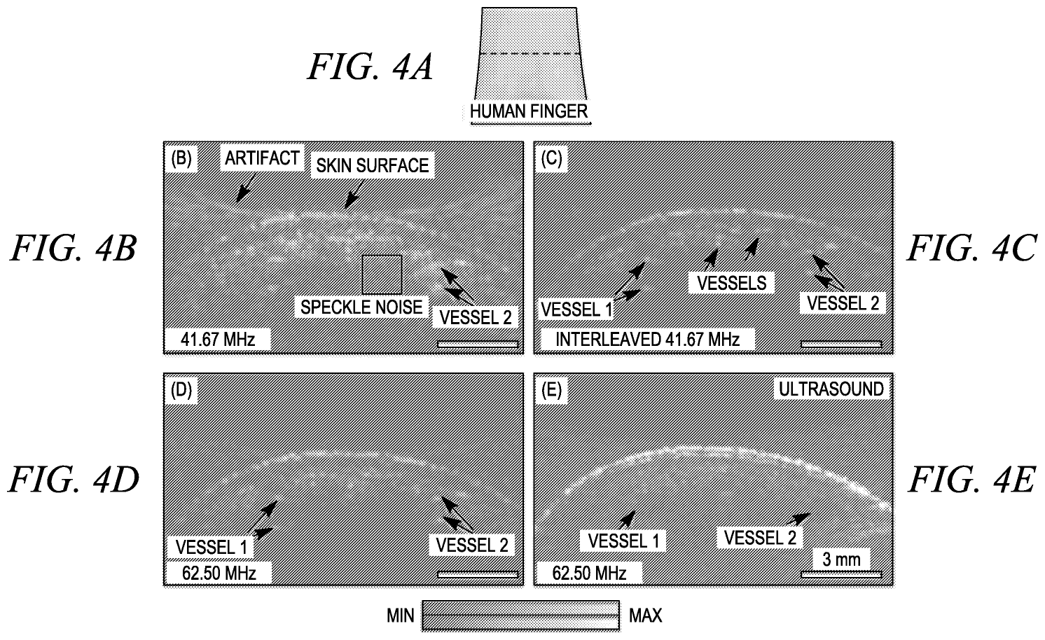

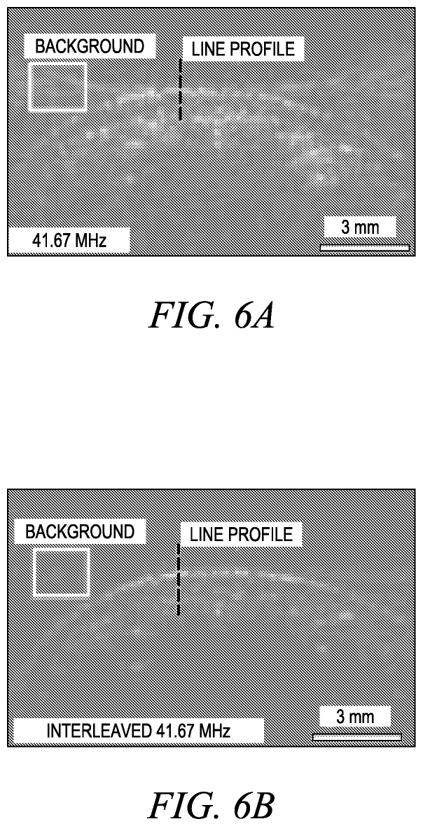

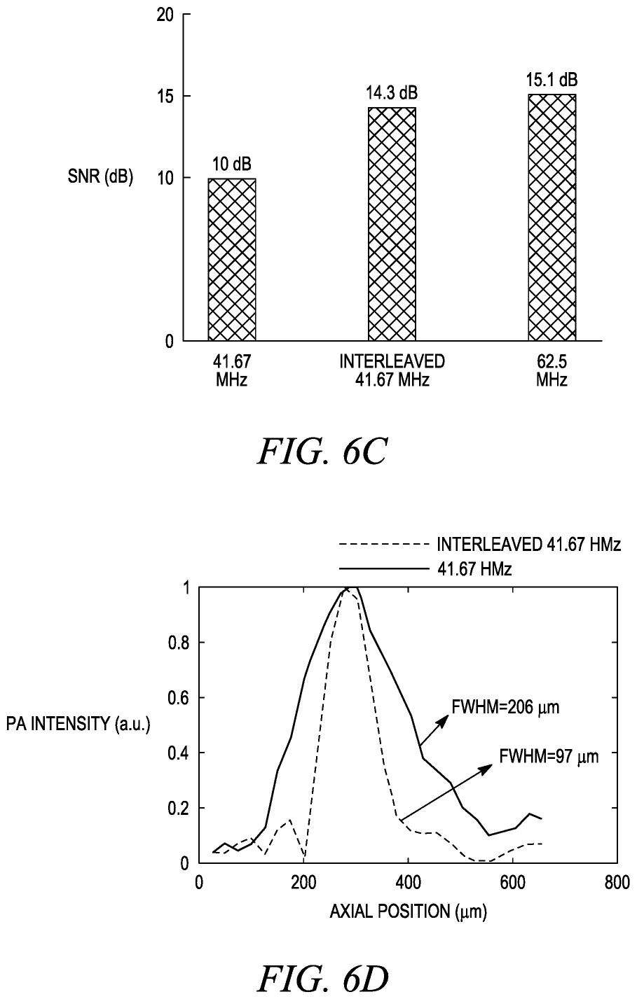

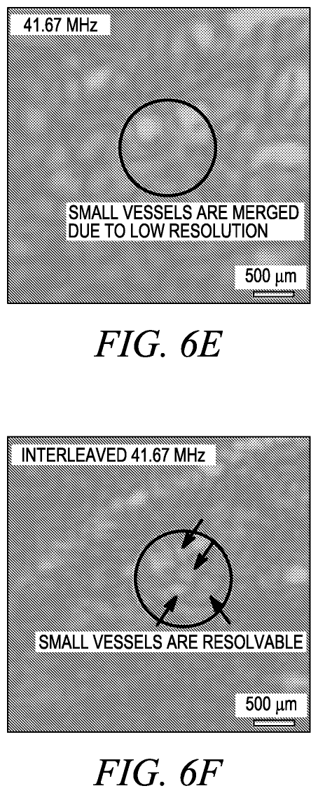

A finger of a healthy volunteer was imaged to further demonstrate the interleave-sampled PA imaging. The finger was held 10 mm underneath the transducer in water. FIG. 4(a) shows the finger. We performed imaging with 41.67-MHz single sampling (FIG. 4(b)), interleaved sampling at 41.67 MHZ (FIG. 4(c)), and 62.5-MHz single sampling (FIG. 4(d)). The frame rate are 20 Hz, 10 Hz, and 20 Hz in FIGS. 4(b), 4(c) and 4(d), respectively. FIG. 4(e) shows the ultrasound image of the finger. Interleaved sampling (FIG. 4(c)) clearly provides more details of blood vessels than the 41.67-MHz sampling (FIG. 4(b)). Most blood signal in FIG. 4(b) is blurred and distorted into large speckles noises, which is a typical issue for high-frequency PA imaging with insufficient sampling rate. A more detailed comparison of the in vivo results is in FIG. 6. Note that the artifact that appeared in the 41.67-MHz sampling (FIG. 4(b)) is because the sampling rate is insufficient; this can be observed in both ultrasound imaging and PA imaging with a linear transducer. There is no significant difference between the 41.67-MHz interleaved sampling and the 62.5-MHz sampling rate (FIG. 4(c) and FIG. 4(d)). This might be because interleaved sampling is subjected to motion. Nevertheless, this in vivo experiment proves that the interleaved sampling method can be used for more high-frequency PA applications when the sampling rate of a DAQ is insufficient.

FIG. 6 compares the in vivo results under 41.67-MHz sampling (FIG. 6a) and interleaved 41.67-MHz sampling (FIG. 6b). A window is defined above the skin surface to extract the background noise to calculate the SNR. FIG. 6c shows the SNR under the three different sampling rates. Interleaved sampling provides higher a SNR than 41.67-MHz sampling (14.3 dB vs 10 dB). (d) The axial PA amplitude distributions across the same skin surface under the two different sampling rates (see the marked line profiles in FIGS. 6a and 6b). Besides hemoglobin, melanin in the skin is another photoacoustic contrast. We evaluate the axial resolution by comparing the line profile across the skin surface. It shows that the interleaved 41.67-MHz sampling provides a much smaller FWHM (97 μm) than the 41.67-MHz sampling (206 μm), which indicates a higher axial resolution. FIGS. 6(e) and 6(f) are amplified PA images of the same imaging region by the 41.67-MHz sampling and interleaved 41.67-MHz sampling, respectively. The circled area has small vessels that are merged into a large speckle and could not be resolved by the 41.67-MHz sampling. In comparison, these small vessels are well separated in the interleaved 41.67-MHz sampling.

There are several factors that may affect the performance of the interleave-sampled PA imaging. For example, interleaved sampling requires multiple acquisitions and needs an accurate delay of the phase of the PA signal. Thus, the temporal resolution (frame rate) becomes worse by a factor of two. Tissue motion can cancel any improvements in resolution. However, this issue is solvable by improving the laser repetition rate. Jitters from the laser and DAQ may also affect the delay. Most state-of-the-art laser systems have a jitter of 1-2 ns, which is sufficiently small for most interleaved sampling. Interleave-sampled PA imaging also requires the two generated PA signals to be as identical as possible as described in FIG. 1(a). Thus, the stability of the light pulse energy is generally important.

In summary, methods and systems described herein have demonstrated the ability of interleave-sampled photoacoustic (PA) imaging for high-frequency imaging with a low sampling rate. The interleaved sampling methods rely on adjusting the laser delay, which can be applied to any PA DAQ systems. This approach allows more ultrasound DAQs, especially clinical DAQs, to be used for high-frequency PA imaging. This lowers the research threshold and costs. Both phantom and in vivo examples experiments were performed with a 30-MHz transducer. Both experiments show that interleaved sampling at 41.67 MHz provides much better imaging quality than the original 41.67-MHz single sampling rate.

FIG. 7 shows a simplified schematic diagram of one example of a PA imaging system 10 in which the methods described herein may be employed. As shown, at least one light source or laser 12, such as an optical parametric oscillator (OPO) laser system, may be used to provide laser pulses. The light source 12. The selection of a light source 12 and the spectrum region in which it operates depends on the imaging purpose, as well known to those of ordinary skill in the art. The light generated by laser 12 may irradiate an imaged sample 14. Pulsed light from the light source 12 may induce photoacoustic signals in an imaged sample 14 that may be detected by a transducer 16, such as an ultrasonic transducer, to generate 2D or 3D photoacoustic tomographic images of the sample 14. The light energy can be delivered to the sample 14 through any methods, such as free space beam path or optical fiber(s). A data acquisition (DAQ) system 18 is provided in communication with laser 12 and transducer 16 and, together with computer 24, comprise a control system. The DAQ system 18 converts the analog signals from the transducer 16 to digital signals. The signals are then sent to a computer 20 and/or cluster of computers which can be a suitable personal computer of sufficient capacity, where the image(s) is reconstructed.

Aspects of the subject matter described herein, such as the sampling of the PA signals and triggers that cause the laser to generate the light pulses, in some cases may be described in the general context of computer-executable instructions, such as computer programs, being executed by a processor. Generally, computer programs include routines, programs, objects, components, data structures, and so forth, which perform particular tasks or implement particular abstract data types. Aspects of the subject matter described herein may also be practiced in distributed computing environments where tasks are performed by remote processing devices that are linked through a communications network. In a distributed computing environment, program modules may be located in both local and remote computer storage media including memory storage devices. For instance, some aspects of the claimed subject matter may be implemented as a computer-readable storage medium embedded with a computer executable program, which encompasses a computer program accessible from any computer-readable storage device or storage media. For example, computer readable storage media can include but are not limited to magnetic storage devices (e.g., hard disk, floppy disk, magnetic strips . . . ), optical disks (e.g., compact disk (CD), digital versatile disk (DVD) . . . ), smart cards, and flash memory devices (e.g., card, stick, key drive . . . ). However, computer readable storage media do not include transitory forms of storage such as propagating signals, for example. Of course, those skilled in the art will recognize many modifications may be made to this configuration without departing from the scope or spirit of the claimed subject matter.

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the embodiments and its practical applications, to thereby enable others skilled in the art to best utilize the embodiments and various modifications as may be suited to the particular use contemplated. Accordingly, the present embodiments are to be considered as illustrative and not restrictive, and the invention is not to be limited to the details given herein, but may be modified within the scope and equivalent of the appended claims.

1. A method of obtaining at least one photoacoustic (PA) image of a sample, comprising:
generating a plurality of light pulses that cause ultrasonic emission from the sample;
receiving ultrasonic emission signals from the sample caused by each of the light pulses;
sampling the ultrasonic emission signals to obtain sampled data values, wherein generating the light pulses includes modulating a time duration between a time at which the light pulses are generated and a time at which the sampling of the ultrasonic emission signals is performed; and
reconstructing at least one photoacoustic image of the sample from the sampled data values of the ultrasonic emission signals.

2. The method of claim 1 wherein the ultrasonic emission signals are sampled at a sampling rate slower than a Nyquist limit.

3. The method of claim 1 wherein the modulating includes offsetting alternating light pulses from an immediately preceding pulse by a time period less than a sampling period.

4. The method of claim 3 wherein the offset is one half of the sampling period.

5. The method of claim 3 wherein the offset is one third of the sampling period.

6. The method of claim 3 wherein the offset is one fourth of the sampling period.

7. The method of claim 3 wherein the plurality of light pulses is generated by an optical source, the modulating being performed by adjusting a time at which a triggering signal is sent to the optical source.

8. The method of claim 1 wherein the plurality of light pulses is generated by a laser source.

9. The method of claim 1 wherein the modulating includes adjusting a timing at which the ultrasonic emission signals are sampled.

10. The method of claim 1 wherein reconstructing the at least one photoacoustic image includes increasing an axial and lateral resolution of the at least one photoacoustic image relative to a photoacoustic image obtained without modulating the time duration such that the light pulses that are generated are equally spaced in time from one another.

11. The method of claim 1 wherein the modulating includes offsetting in time, by a time period less than a sampling period, a subset of the light pulses relative to the time at which the sampling of the ultrasonic signals is performed.

12. The method of claim 11 wherein the offsetting includes advancing in time the subset of the light pulses relative to the time at which the sampling of the ultrasonic signals is performed.

13. The method of claim 11 wherein the offsetting includes delaying in time the subset of the light pulses relative to the time at which the sampling of the ultrasonic signals is performed

14. The method of claim 11 wherein the offsetting includes advancing in time a first subset of the light pulses relative to the time at which the sampling of the ultrasonic signals is performed and delaying in time a second subset of the light pulses relative to the time at which the sampling of the ultrasonic signals is performed.

15. The method of claim 11 wherein different ones of the light pulses in the subset of the light pulses are offset by different fractions of the sampling period.

16. The method of claim 11 wherein the subset of the light pulses are alternating ones of the light pulses.

17. A non-transitory computer-readable medium, comprising instructions for causing a computing device to cause the method of claim 1 to be performed.
